(19) United States
(12) Patent Application Publication
(10) Pub. No.: US 2017/0156623 A1
(43) Pub. Date: Jun. 8, 2017

(54) SELF-ADHESIVE MICROFLUIDIC AND SENSOR DEVICES

(71) Applicant: The Regents of the University of California, Oakland, CA (US)

(72) Inventors: Michael Chu, Irvine, CA (US); Thao Nguyen, Irvine, CA (US); Michelle Khine, Irvine, CA (US); Eugene Lee, Irvine, CA (US)

(21) Appl. No.: 15/373,353

(22) Filed: Dec. 8, 2016

Related U.S. Application Data
(60) Provisional application No. 62/264,769, filed on Dec. 8, 2015.

(51) Int. Cl. A61B 5/0408; A61B 5/0478; A61B 5/0492; B01L 3/00

(52) U.S. Cl. CPC A61B 5/04087; B01L 3/502707; A61B 5/0478; A61B 5/0492; B01L 2300/16; B01L 2200/12; B01L 2200/0689; B01L 2200/027; A61B 2562/0209; A61B 2562/028; A61B 2562/125; A61B 2562/168

(57) ABSTRACT

Methods of forming a microfluidic device include: combining a volume of uncured liquid silicone based polymer with a volume of adhesive polymer to provide a flowable material; applying the flowable material to a mold and curing the flowable material on the mold to form a microfluidic device layer comprising an exposed face with at least one channel or chamber; and contacting the exposed face of the microfluidic device layer to a substrate to adhere the microfluidic device layer to the substrate to enclose the at least one channel or chamber to form a microfluidic device. Other methods include combining a volume of uncured liquid silicone based polymer with a volume of adhesive polymer to provide an intermediary material; applying a layer of the intermediary material to a substrate and curing the layer of the intermediary material on the substrate; obtaining a silicon based polymer that comprises an exposed face that comprises at least one channel or chamber; and contacting the exposed face of the silicon based polymer to the cured layer of the intermediary material, wherein the exposed face of the silicon based polymer adheres to the cured layer of the intermediary material to enclose the at least one channel or chamber to form a microfluidic device. Also disclosed are microfluidic devices and sensors comprising the microfluidic devices.

### BACKGROUND OF THE INVENTION

Field of the Invention

The application is directed to microfluidic devices methods of assembling microfluidic devices and sensor devices made from the microfluidic devices.

Description of the Related Art

Fabrication of a functional microfluidic device necessitates a substantial seal between the device and substrate for leak-proof encapsulation of the channels and chambers. This crucial step has been the focus for developing novel and versatile bonding techniques. While there are many different materials used for fabricating microfluidic chips, replica molding with polydimethylsiloxane (PDMS) is currently one of the most common prototyping procedure (E. J. Sackmann, A. L. Fulton and D. J. Beebe, Nature, 2014, 507, 181); however, as PDMS does not readily adhere to most substrates, an adhesion step is required to strongly bond the PDMS device and substrate together. The ubiquitous method for sealing PDMS-based devices is via oxygen plasma treatment of both the PDMS and the substrate's surfaces before placing them in contact with each other immediately after activation. Oxygen plasma treatment activates the surfaces of both the PDMS device and glass substrate by replacing Si—CH₃ bonds with Si—OH groups. The surfaces bond irreversibly when the reactive —OH groups are put in contact with each other, forming a covalent Si—O—Si bond between the glass and the PDMS (M. J. Owen, P. J. Smith and J. Adhesion Sci. Technol., 1994, 8, 1063). Although this process produces a strong and irreversible seal, it is a time sensitive step and necessitates access to an oxygen plasma machine. Moreover, this bonding method limits throughput because of the time dependency of the surface activation and the limited size of a typical oxygen plasma chamber. Additionally, once contact between the activated surfaces is made, removing the surfaces is no longer possible, making microfluidic chips that require tight alignment tolerances, such as 3D devices, difficult. Due to these limitations, alternate methods have been developed for irreversibly sealing microfluidic chips on glass and alternative substrates. For example, popular alternatives include utilizing: corona treatment, partially cured PDMS, or chemical cross-linkers (K. Haubert, T. Drier and D. Beebe, Lab Chip, 2006, 6, 1548; H. Wu, B. Huang and R. N. Zare, Lab Chip, 2005, 5, 1393; L. Tang and N. Y. Lee, Lab Chip, 2010, 10, 1274; and W. Wu, J. Wu, J.-H. Kim and N. Y. Lee, Lab Chip, 2015, 15, 2819).

While irreversible bonding is often sufficient for many microfluidic operations, there are certain circumstances where a reversible seal is advantageous (Y. Temiz, R. D. Lovchik, G. V. Kaigala and E. Delamarche, Microelectronic Engineering, 2015, 132, 156). For instance, in cell culture systems, where subsequent harvesting of the cell or tissue sample is required, easy access to the channels is desirable. However, research focused on reversible microfluidic bonding is limited (Y. Temiz, R. D. Lovchik, G. V. Kaigala and E. Delamarche, Microelectronic Engineering, 2015, 132, 156), with many of these methods requiring extra components or processing to create a reversible seal (E. Tkachenko, E. Gutierrez, M. H. Ginsberg and A. Groisman, Lab Chip, 2009, 9, 1085; A. Lamberti, A. Sacco, S. Bianco, E. Guiri, M. Quaglio, A. Chiodoni and E. Tresso, Microelectronic Engineering, 2011, 88, 2308; and A. Wasay and D. Sameoto, Lab Chip, 2015, 15, 2749). Alternatively, simpler sealing methods have also been proposed. Thompson et al. used double-sided tape to seal their PDMS devices (C. S. Thompson and A. R. Abate, Lab Chip, 2013, 13, 632). They reported a bonding method that can withstand high-pressure operation. More recently, Shiroma et al. have reported a simple sandwich bonding method that produces a strong seal by sandwiching a glass coverslip against the channels with PDMS (L. S. Shiroma, M. H. O. Piazzetta, G. F. Duarte-Junior, W K. T. Coltro, E. Carrilho, A. L. Gobbi and R. S. Lima, Scientific Reports, 2016, 6, DOI: 10.1038/srep26032).

Overall, methods for creating irreversibly or reversibly sealed microfluidic devices typically require capital equipment or specialized components, adding complication to the fabrication process. While fabrication of single layer devices is achievable with the aforementioned methods, the process for creating more specialized chips, such as multilayers devices or channels and chambers with functionalized surfaces, becomes more difficult. For example, any surface modification made on the microfluidic channels, chambers, or substrate must be able to withstand the subsequent bonding procedure used afterwards.

This need for compatibility between the adhesion layer and surface modification is exemplified with cell patterning within a sealed fluidic chamber. Micropatterning is one of the most widely used methods to spatially grow cells in a deterministic pattern; when combined with a microfluidic environment, it allows for greater control and manipulation of the cells (N. K. Inamdar and J. T. Borenstein, Current Opinion in Biotechnology, 2011, 22, 681). Micropatterning is normally achieved by functionalizing the surface of the substrate in a specified pattern for cell adhesion; however, it is difficult to pattern cells within a fluidic device because the compatibility between the patterned area and the bonding step must be considered. While many have reported methods on the micropatterning of open substrates (S. A. Ruiz and C. S. Chen, Soft Matter, 2007, 3, 168; Z. Nie and E. Kumacheva, Nature Materials, 2008, 7, 277; R. S. Kane, S. Takayama, E. Ostuni, D. E. Ingber and G. M. Whitesides, Biomaterials, 1999, 20, 2363; and X. Mu, W. Zheng, J. Sun, W. Zhang and X. Jiang, Small, 2013, 9, 9), there have been relatively few reported methods for micropatterning within a fluidic device (L. Wang, L. Lei, X. F. Ni, J. Shi and Y. Chen, Microelectronic Engineering, 2009, 86, 1462; A. Khademhosseini, J. Yeh, G. Eng, J. Karp, H. Kaji, J. Borenstein, 0. C. Farokhzad and R. Langer, Lab Chip, 2005, 5, 1380; and S. W. Rhee, A. M. Taylor, C. H. Tu, D. H. Cribbs, C. W. Cotman and N. L. Jeon, Lab Chip, 2005, 5, 102). Moreover, the reported methods are often laborious, multistep processes meant for laboratories specialized in microfluidics, which greatly limits accessibility of this technology to general laboratories. Having a simple fabrication method without an additional adhesion layer would not only provide greater versatility of the device for cell research, but also increase accessibility of the platform to non-specialized laboratories.

A key to fabricating successful microfluidic devices is too strongly seal the device to a substrate (i.e., PDMS to glass). However, the most common device material, PDMS, requires additional processing in order to effectively bond the device channels to the substrate. Typical methods are: oxygen plasma treatment, Carona treatment, partially cured PDMS bonding, use of a chemical crosslinker, or applying double stick tape to the surface. While oxygen plasma is the most effectual and widely used method (by functionalizing the PDMS surface and creating strong bonds), it requires both expensive specialization equipment and potentially clean room access. All other methods are extensively time consuming and introduce additional complications the fabrication process while remaining relatively ineffectual.

### SUMMARY OF THE INVENTION

We created a silicon-based polymer mixture that can adhere directly onto glass and other substrates. The polymer is used in place of traditional PDMS to mold microfluidic chips. Due to its high adhesion, the self-adhesive polymer can be placed directly onto a glass substrate (e.g., a glass slide) to enclose the channels once it is fully cured, resulting in a reversibly bond between the cured self-adhesive polymer and the substrate. This fabrication method does not require any type of surface treatment of the polymer in order to bond to it to glass. Furthermore the microfluidic chips can be peeled off, washed and reused. This type of microfluidic device has potentially exciting applications as the polymer can be directly adhered to the skin, allowing us to use microfluidics on the skin surface. In some embodiments, the microfluidic device is placed on the skin surface, but the fluid is not in direct contact from within the channel. In other embodiments, the microfluidic device is placed on the skin and the fluid can be in direct contact with the skin surface from within the channel.

Using a self-adhesive polymer, we can also achieve irreversible bonding within microfluidic chips that can withstand much higher pressures compared to the reversible bonding that we disclose herein. Compared to conventional methods, our fabrication method is more versatile and simpler and does not require any capital equipment or clean room access.

Some embodiments relate to a method of forming a microfluidic device, comprising:

combining a volume of uncured liquid silicone based polymer with a volume of adhesive polymer to provide a flowable material;

applying the flowable material to a mold and curing the flowable material on the mold to form a microfluidic device layer comprising an exposed face with at least one channel or chamber; and

contacting the exposed face of the microfluidic device layer to a substrate to adhere the microfluidic device layer to the substrate to enclose the at least one channel or chamber to form a microfluidic device.

In some embodiments, a ratio of the volume of the uncured liquid silicone based polymer to the volume of the adhesive polymer is at least 1:10, 1:20, 1:30, 1:40, 1:60 and/or does not exceed 1:100.

In some embodiments, the mold comprises a positive mold.

In some embodiments, the curing comprises heating the flowable material.

In some embodiments, the heating comprises heating for 2 hours at 60° C.

In some embodiments, the curing comprises applying a vacuum to the flowable material.

In some embodiments, the silicone comprises PDMS and the adhesive polymer comprises a soft-skin adhesive.

Some embodiments further comprise forming an inlet to the microfluidic device by creating a passage through at least one of the microfluidic device layer and the substrate.

In some embodiments, the passage is created through the microfluidic device layer prior to adhering the exposed face to the substrate.

In some embodiments, the substrate is micropatterned to create functionalized patterns on the substrate to contacting the exposed face of the microfluidic device layer to a substrate.

In some embodiments, the substrate is micropatterned by microcontact printing.

Some embodiments relate to a method of forming a microfluidic device, comprising:

combining a volume of uncured liquid silicone based polymer with a volume of adhesive polymer to provide an intermediary material;

applying a layer of the intermediary material to a substrate and curing the layer of the intermediary material on the substrate;

obtaining a silicon based polymer that comprises an exposed face that comprises at least one channel or chamber; and

contacting the exposed face of the silicon based polymer to the cured layer of the intermediary material, wherein the exposed face of the silicon based polymer adheres to the cured layer of the intermediary material to enclose the at least one channel or chamber to form a microfluidic device.

In some embodiments, a ratio of the volume of the uncured liquid silicone based polymer to the volume of the adhesive polymer is at least 1:10, 1:20, 1:30, 1:40 or 1:60 and/or does not exceed 1:100.

In some embodiments, the mold comprises a positive mold.

In some embodiments, the curing comprises heating the layer of the intermediary material on the substrate.

In some embodiments, the heating comprises heating for 2 hours at 60° C.

In some embodiments, the curing comprises applying a vacuum to the layer of the intermediary material on the substrate.

In some embodiments, the silicone comprises PDMS and the adhesive polymer comprises a soft-skin adhesive.

Some embodiments further comprise forming an inlet to the microfluidic device by creating a passage through at least one of the silicon based polymer nd the substrate.

In some embodiments, the passage is created through the silicon based polymer prior to adhering the exposed face to the substrate.

In some embodiments, the substrate layer is micropatterned to create functionalized patterns on the substrate prior to contacting the exposed face of the silicon based polymer to a substrate.

In some embodiments, the substrate is micropatterned by microcontact printing.

Some embodiments relate to a microfluidic device comprising:

a first substrate layer, and

a second layer comprising a silicone based polymer and an adhesive polymer, wherein the second layer comprises at least one channel or chamber at a surface of the second layer,

wherein the first substrate layer and the second layer are adhered together to enclose the at least one channel or chamber within the microfluidic device.

Some embodiments relate to a microfluidic device comprising:

a first substrate layer,

a second intermediary layer that comprises a silicone based polymer and an adhesive polymer, and

a third layer comprising a silicon based polymer that comprises at least one channel or chamber at a surface of the third layer;

wherein the first substrate layer is adhered to the second intermediary layer and the third layer is adhered to the second intermediary layer, wherein the at least one channel or chamber at the surface of the third layer is enclosed within the microfluidic device.

Some embodiments relate to a sensor comprising a microfluidic device layer comprising a silicone based polymer and an adhesive polymer, the microfluidic device layer comprising an exposed face that is configured to adhere directly to skin of a user or patient.

In some embodiments, the microfluidic device is placed on a skin surface, fluid in a channel in the microfluidic device does not contact the skin surface.

In other embodiments, the microfluidic device is placed on a skin surface, fluid in a channel in the microfluidic device does not contact the skin surface.

In one embodiment, a method is provided for forming a microfluidic device. A volume of uncured liquid PDMS, other silicone based polymer, or another biocompatible and/or inert polymer is provided. A volume of adhesive polymer is also provided. The silicone based polymer or other biocompatible and/or inert polymer and the adhesive polymer are combined, e.g., in a ratio of at least 1:10 biocompatible and/or inert polymer to adhesive polymer. The combination provides a flowable microfluidic device material. The flowable microfluidic device material is applied to a mold. The flowable microfluidic device material is cured on the mold to form a microfluidic device layer. The layer includes an exposed face with at least one channel or chamber. The exposed face of the microfluidic device layer is adhered to a substrate to enclose the at least one channel or chamber to form a microfluidic device.

In another embodiment, a method of using a microfluidic device is provided. In the method a microfluidic device layer and a substrate are provided. The microfluidic device layer comprises (e.g., is made of) an inert polymeric material and a self-adhesive polymer. The microfluidic device has an exposed face having at least one channel or chamber formed therein. The exposed face of the microfluidic device is coupled with the substrate to enclose the at least one channel or chamber.

In some further methods, a substance is then flowed through the at least one channel in connection with a diagnostic procedure, an analysis, or other study of the substance.

In another embodiment, a microfluidic sample handling apparatus is provided. The microfluidic sample handling apparatus includes a microfluidic device layer and a substrate. The microfluidic device layer has, e.g., is made from, an inert polymeric material and a self-adhesive polymer. The microfluidic device has a first exposed face having at least one channel or chamber disposed therein. The substrate has a second exposed face. The first exposed face is configured to adhere directly to the second exposed face in order to enclose the at least one channel.

In another embodiment, a skin adhesive layer comprising a diagnostic apparatus is provided.

The diagnostic apparatus can comprise in one class of devices a microfluidic sample handling apparatus includes a microfluidic device layer. The microfluidic device layer has, e.g., is made from, an inert polymeric material and a self-adhesive polymer. The microfluidic device has a first exposed face having at least one channel or chamber disposed therein. The first exposed face is configured to adhere directly to the skin of a user or patient in order to enclose the at least one channel between the layer and the skin.

The diagnostic apparatus can comprise in another class of devices a sensor, such as a dry electrode sensor or biopotential sensor. Examples of such sensors include an EKG sensor for sending heart parameter, EMG sensor for sensing muscular activity and parameter, and an EEG sensor for sensing brain activity and parameters. The sensor apparatuses can comprise a sensor layer. The sensor layer has, e.g., is made from, an inert polymeric material and a self-adhesive polymer. The sensor layer has an exposed face. At least one sensing device is disposed in the layer, e.g., fully encapsulated therein or at the exposed face. The exposed face is configured to adhere directly to the skin of a user or patient in order to bring the sensor into sufficiently close adjacency, e.g., touching, the skin.

### BRIEF DESCRIPTION OF THE DRAWINGS

These and other features, aspects and advantages are described below with reference to the drawings, which are intended to illustrate but not to limit the inventions. In the drawings, like reference characters denote corresponding features consistently throughout similar embodiments. The following is a brief description of each of the drawings.

FIG. 1. (a) Image of a 3D micromixer. (b) Schematic of the different layers. The top and bottom layer were molded using PDMS while the middle layer was molded with the Adhesive Polymer. The device was assembled on a glass slide coated with cured adhesive polymer.

FIG. 2. (a) Process flow for fabricating the reversibly sealed device. (i) A master mold was first casted with adhesive polymer. (ii) The polymer was then cured at 60 degrees Celsius. (iii) Afterwards, the polymer was removed and placed onto a clean glass substrate. (iv) The construct was then heated at 120 degrees Celsius for 90 minutes. (b) Process flow for fabricating the permanently sealed device. (i) A thin layer of the uncured adhesive polymer was first spun coat onto a glass slide. (ii) The polymer was then cured at 60 degrees Celsius. (iii) A traditionally casted PDMS microfluidic mold was placed onto the cured adhesive polymer substrate. (iv) The entire device was heat treated at 120 degrees Celsius for 90 minutes.

FIG. 3 (a) Fabrication process for permanently sealed microfluidic devices. (b) Removal of a PDMS chamber that has been permanently sealed. The rough texture is the tearing of the self-adhesive polymer.

FIG. 4 shows a process of preparing a microfluidic device layer.

FIG. 5(a) shows a microfluidic sample handling apparatus including a microfluidic device layer comprising an inert and/or biocompatible polymer and an adhesive component;

FIG. 5(b) shows certain features of a microfluidic device layer.

FIG. 6(a) shows the application of a microfluidic device layer to a substrate to enclose at least one channel thereof;

FIG. 6(b) shows fluid flowing through several channels of a microfluidic device formed with a self-adhesive microfluidic device layer;

FIG. 6(c) shows a step of removing the microfluidic device layer from the substrate;

FIG. 6(d) shows the cleaning of the microfluidic device layer for subsequent re-use;

FIG. 6(e) shows that after the microfluidic device layer has been cleaned it can be re-adhered to a new substrate for additional use(s).

FIG. 7 shows the application of a skin adhesive layer apparatus comprising a diagnostic apparatus, such as a microfluidic device layer directly onto the skin of a user or patient.

FIG. 8. Schematic of a pressure burst set up assembly. Inlet tubing goes through a press fit tubing connector.

FIG. 9. (a) Cross sectional diagram of the three test conditions for the pressure burst test. PDMS adhered directly onto the glass slide served as the control. (b) Graph of the last stable pressure before bond failure occurred for each of the conditions.

FIG. 10. (a) Fluid chamber filled with blue dye. (b) Removal of the irreversibly sealed PDMS. (c) Top down view of the adhesive polymer substrate post removal. The inset image shows a magnified view of the cell chamber border between bonded and non-bonded areas. Removal of the PDMS chamber ripped the adhesive polymer layer (right side of the inset image) while leaving the substrate within the chamber intact (left side of the inset image).

FIG. 11. Percent swelling of PDMS and the Adhesive Polymer in various solvents. As described below, the initial length of each side for each of the pieces was measured immediately upon submersion into the solvent. After a 24 hour period to allow the swelling to reach equilibrium, the length of each side was again measured. The difference between the two lengths was then normalized by the initial length in order to obtain a percent change in length for each respective solvent. The swelling of PDMS and the Adhesive Polymer was found to be statistically insignificant from each other for each solvent (Acetone: p=0.6311, IPA: p=0.1509, Ethanol: p=0.4849, DMSO: p=0.8725, Water: p=0.2154).

FIG. 12. (a) Reversibly sealed microfluidic gradient generator with blue and yellow food dye. (b-d) Sequence for removal of the adhesive polymer device from the glass substrate after second use.

FIG. 13. (a) Process flow for sealing the micropatterned substrate within a PDMS device. (i) The PDMS device is molded via replica molding, and the substrate is made by depositing a layer of adhesive polymer over a glass slide via spin coating. (ii) The micropattern is formed on the cured adhesive polymer substrate. (iii) The PDMS device is sealed against the substrate through direct contact. (iv) Cells are loaded into the construct. (b) Two patterned square islands with live cardiomyocytes. (c) Motion vectors (red arrows) of the cardiomyocyte contractions generated using optical flow. (d) Graph of the first PCA from the optical flow.

### DETAILED DESCRIPTION OF CERTAIN EMBODIMENTS

This application is directed to convenient microfluidic devices and methods for making such devices. The methods create devices more quickly and efficiently and expand the usefulness of such devices. For example, we have a novel, innovative approach toward molding microfluidic channels, in that we use a self-adhesive polymer mixture. The polymers we use enable the devices to effectively seal to a substrate without additional surface treatment.

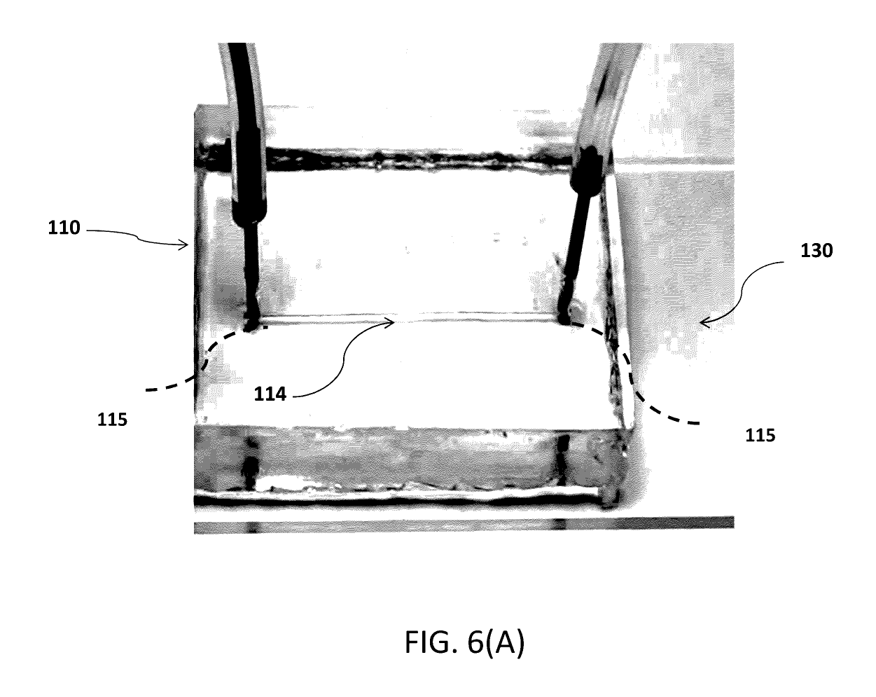

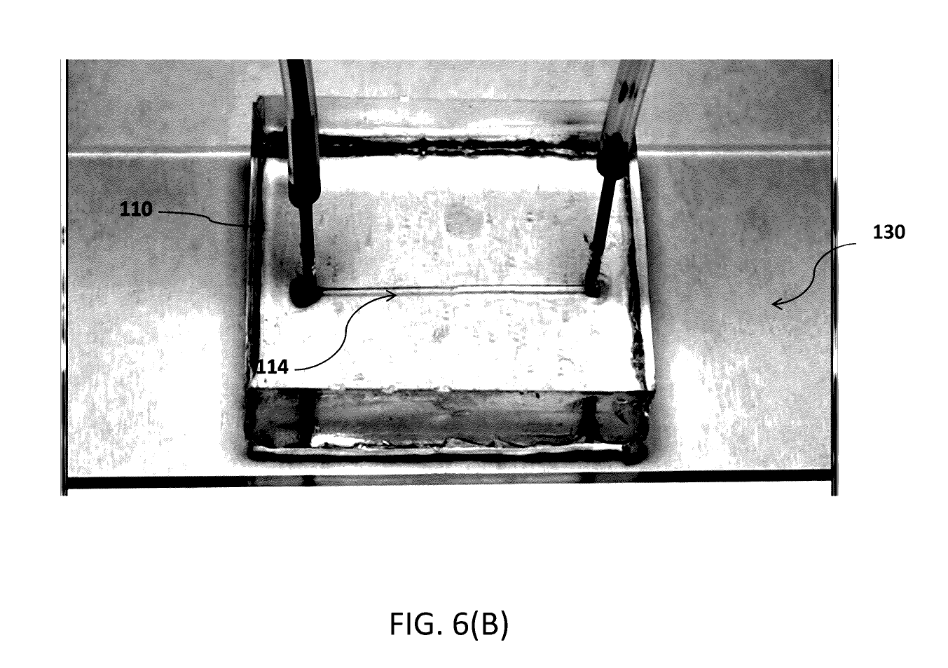

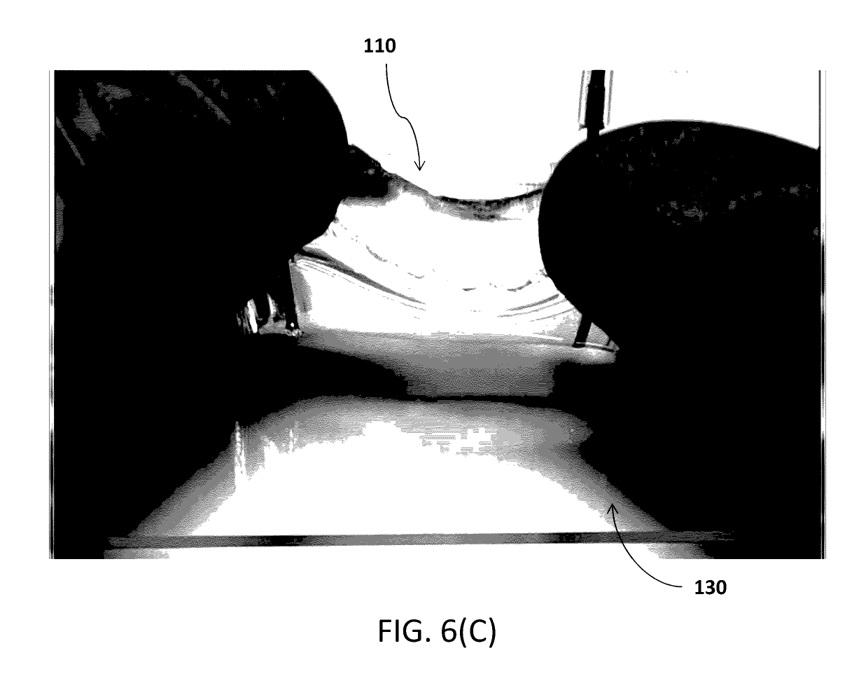

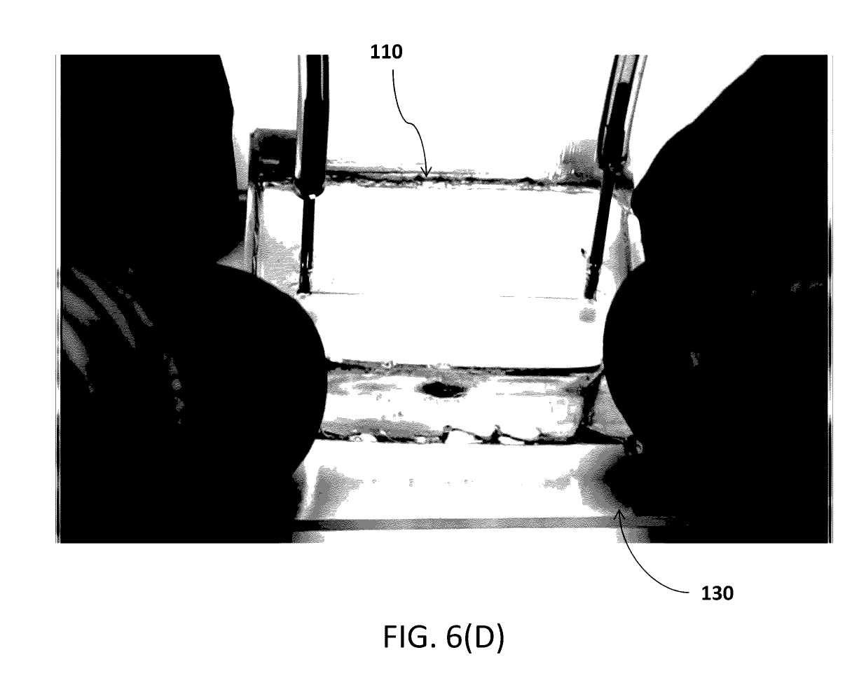

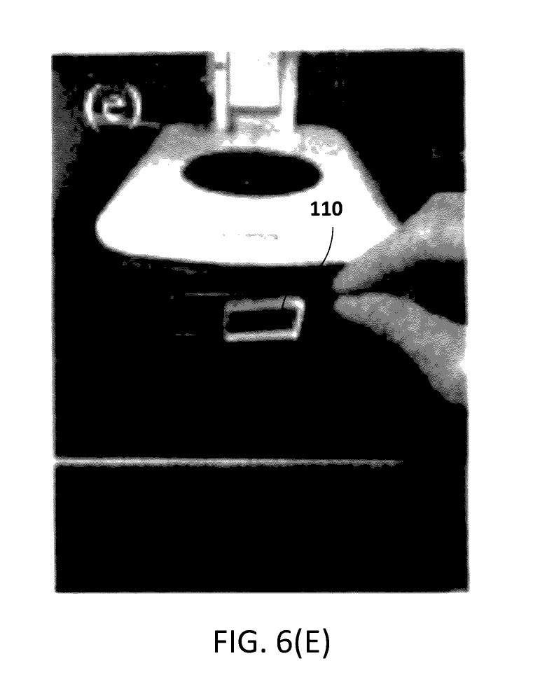

The self-adhesive polymer is made from the ratio standard PDMS (e.g., Dow Corning Sylgard 184) to soft skin adhesive (e.g., Dow Corning MG7-9850). Ratios of 1:20, 1:30, 1:40, 1:50, and 1:60 (PDMS:MG7-9850) have been mix to create a self-adhesive polymer of different stiffness and tackiness. Ratios containing larger amounts of MG7-9850 may be softer and tackier. For our purposes, we have demonstrated successful fabrication of microfluidic chips with the ratio of 1:40. We successfully created a gradient generating microfluidic device depicted in FIG. 6, A and B using a self-adhesive polymer.

We demonstrate a simple and versatile plasma free bonding method that can achieve both a reversible and irreversible seal with microfluidic devices. Following convention, we choose to define irreversible bonding as a seal that can withstand greater than 207 kPa (S. K Sia and G. M. Whitesides, Electrophoresis, 2003, 24, 3563) in which the polymer surface is compromised upon removal; a reversible seal, on the other hand, allows for the device to be removed and then reapplied without any damage. Our process allows for facile fabrication of multilayer PDMS devices while also being compatible with micropatterning technique for patterned cell growth within a fluidic chamber.

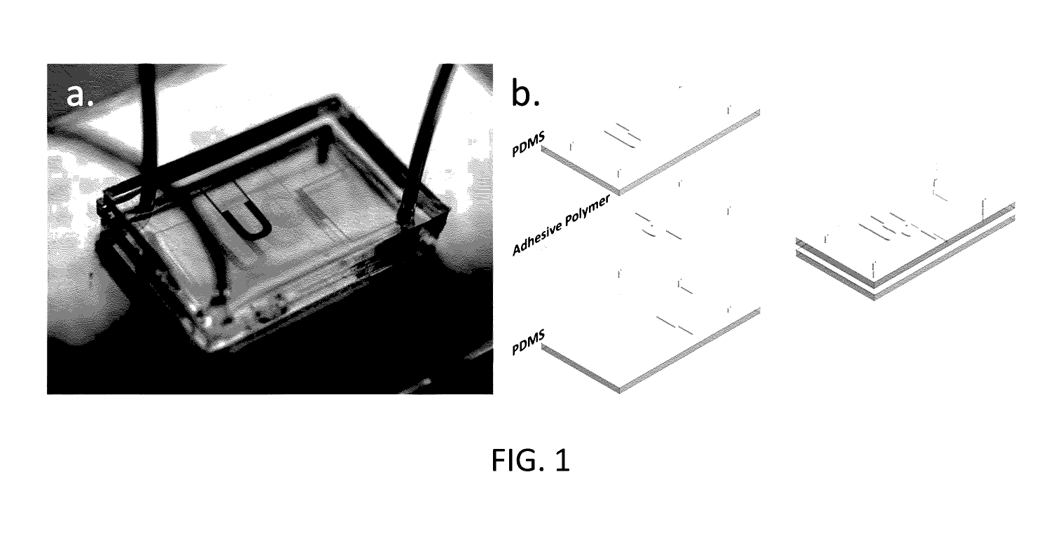

Instead of applying an adhesive layer to bond the PDMS device and substrate together, we use a PDMS-based adhesive polymer as the substrate for direct adhesion of PDMS devices. The adhesive polymer can also be used to mold microfluidic devices. When cured, the polymer mixture exhibits high adhesion, which is leveraged as a sealing mechanism for a reversible seal against glass. Conversely, an irreversible bond can be achieved between the cured adhesive polymer and PDMS after a simple heat treatment of the two polymers in contact with each other. We applied the adhesive polymer with PDMS to demonstrate a facile process for fabricating an irreversibly bonded multilayer 3D microfluidic device (FIG. 1a-b); we also show the fabrication of a reversibly sealed device against glass. Lastly, we demonstrate the compatibility of this system with micropatterning by creating a large array of square islands for cell culturing within a fluidic chamber. Importantly, with this approach, laboratories and classrooms without any capital equipment can easily fabricate a larger variety of microfluidic devices.

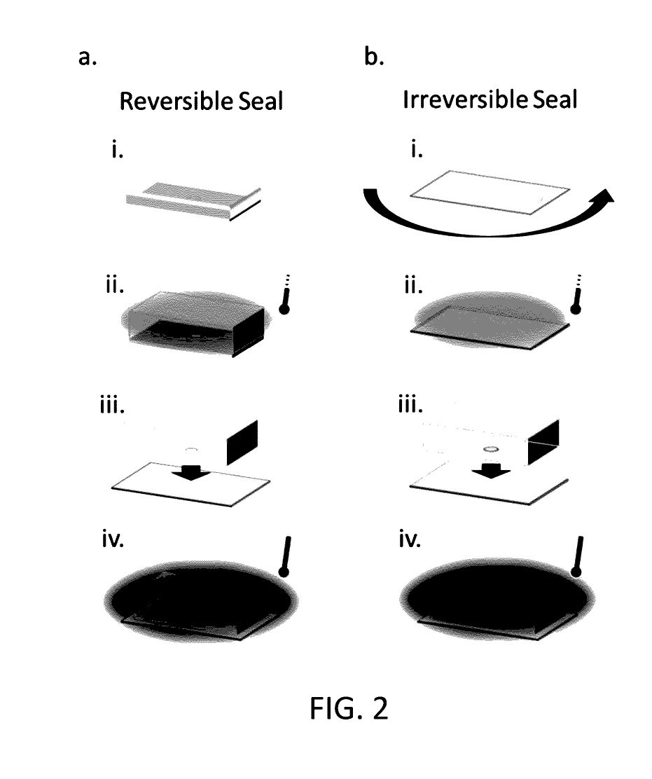

The adhesive polymer is a mixture of a silicone-based soft skin adhesive and traditional PDMS. Both polymers are first mixed separately and then combined to form the final adhesive mixture. The PDMS is prepared by mixing the cross linker and base, and the soft skin adhesive was prepared by mixing part A and part B components. The adhesive polymer mixture is then formed by combining the uncured PDMS and soft skin adhesive. Next, the final adhesive mixture is used to mold the microfluidic devices following the traditional replica molding process (D. C. Duffy, J. C. McDonald, O. J. A. Schueller and G. M. Whitesides, Anal. Chem., 1998, 70, 4974), cured, and bonded to a glass substrate for a reversible seal (FIG. 2a). Alternatively, for an irreversible bond, cured adhesive polymer was spun coat onto a glass slide and cured; the cured adhesive polymer was then used as a substrate to bond traditionally molded PDMS devices (FIG. 2b). The PDMS device may be placed directly on the cured adhesive polymer substrate, and heat treated to create an irreversible seal.

### Reversible Bonding

To establish a reversible bond between a device and a substrate (e.g., a clean glass substrate), a master mold is first casted with the self-adhesive polymer. The polymer is then cured, e.g., at 60° C. Application of heat accelerates the curing process of the polymer. In some embodiments, the polymer may be cured at temperatures ranging from 0° C. to 150° C., including room temperature and temperatures of about 0° C., 10° C., 20° C., 30° C., 40° C., 50° C., 60° C., 70° C., 80° C., 90° C., 100° C., 110° C., 120° C., 130° C., 140° C. and 150° C. Afterwards, the polymer is removed and placed onto a clean glass substrate. The construct is then heated, e.g., at 120° C. for 90 minutes, thereby establishing a reversible seal between the cured self-adhesive polymer and the substrate. In some embodiments, the reversible seal may be established at temperatures ranging from 0° C. to 150° C., including room temperature and temperatures of about 0° C., 10° C., 20° C., 30° C., 40° C., 50° C., 60° C., 70° C., 80° C., 90° C., 100° C., 110° C., 120° C., 130° C., 140° C. and 150° C.

The reversible bond between the cured device cast from the self-adhesive polymer and a substrate is sufficiently strong to withstand pressures of about 79±5 kPa. In some embodiments, the reversible bond can withstand pressures of up to 50 kPa, 55 kPa, 60 kPa, 65 kPa, 70 kPa, 75 kPa, 80 kPa, 85 kPa, 90 kPa, 95 kPa or 100 kPa. By comparison, a reversible bond established between a conventional device made from PDMS and a substrate exhibits a lower failure pressure on the order of about 20 kPa.

A benefit of having a reversible bond is that a device may be cleanly removed from the substrate and reused. The device and substrate are held together by van der Waals adhesion.

Irreversible Bonding with Increased Bond Strength

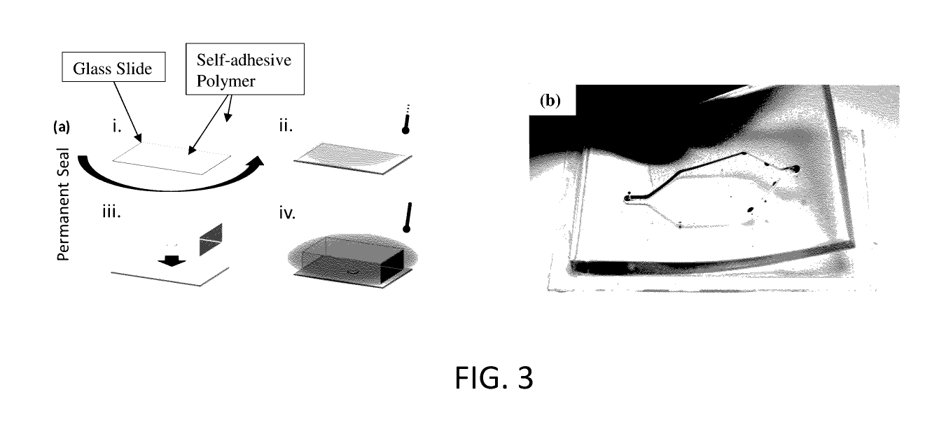

In addition to the reversible bonding described above, an irreversibly bonded microfluidic device can also be achieved by using the self-adhesive polymer as an adhesion layer for traditionally molded PDMS microfluidic devices. FIG. 3a shows the procedure for fabricating the irreversibly sealed devices. The self-adhesive polymer is spun coat onto a glass slide (at a thickness of approximately 50 μm) and cured. In some embodiments, the polymer may be cured at temperatures ranging from 0° C. to 150° C., including room temperature and temperatures of about 0° C., 10° C., 20° C., 30° C., 40° C., 50° C., 60° C., 70° C., 80° C., 90° C., 100° C., 110° C., 120° C., 130° C., 140° C. and 150° C. Afterwards, a cured PDMS microfluidic device is placed in contact with the cured self-adhesive polymer/glass substrate before a heat treatment is applied to create an irreversible bond. In some embodiments, the irreversible seal may be established by heating at temperatures ranging from 0° C. to 150° C., including room temperature and temperatures of about 0° C., 10° C., 20° C., 30° C., 40° C., 50° C., 60° C., 70° C., 80° C., 90° C., 100° C., 110° C., 120° C., 130° C., 140° C. and 150° C. This sealing method is also compatible with traditional PDMS microfluidic device process flow. It does not require any capital equipment or clean room access for the sealing.

An irreversible bond between a conventional cured PDMS device and a substrate is sufficiently strong to withstand pressures of about 207-345 kPa. In some embodiments, the irreversible bond can withstand pressures of up to 207 kPa, 210 kPa, 220 kPa, 230 kPa, 240 kPa, 250 kPa, 260 kPa, 270 kPa, 280 kPa, 290 kPa, 300 kPa, 310 kPa, 320 kPa, 330 kPa, 340 kPa or 350 kPa.

The self-adhesive polymer is softer than the PDMS, and when a tensile stress is applied to remove the PDMS device, the self-adhesive substrate mechanically fails before the PDMS does. This results in the self-adhesive polymer substrate tearing, allowing the PDMS to be pulled off (see FIG. 3b).

An example fabrication of a permanently bonded microfluidic device is as follows:
1. PDMS (Sylgard 184) is made by mixing the cross-linker and base at a ratio of 1:10 by weight.
2. The soft skin adhesive polymer (MG7-9850) is made by mixing the part A and part B components at a ratio of 1:1 by weight.
3. The PDMS and MG7-9850 are mixed together at the desired ratio (1:40 of the PDMS to MG7-9850 respectively) for form the uncured self-adhesive polymer.
4. The uncured self-adhesive polymer is allowed to degas in vacuum for 10 minutes.
5. The uncured self-adhesive polymer is then spun coat onto a glass slide (FIG. 3a-i).
   a. The glass slide may be predrilled with inlet holes depending on application
   b. The spin speed can vary depending on how thick you want the self-adhesive polymer layer. We had a 50 μm layer.
6. The glass slide with the uncured self-adhesive polymer is then cured at 60 degrees Celsius for 3 hours (FIG. 3a-ii). At this stage, the cured self-adhesive polymer does not feel extremely tacky.
7. A PDMS microfluidic device is fabricated using the traditional soft lithographic fabrication method.
8. The cured PDMS microfluidic device is placed (channel side facing down) on to the cured self-adhesive substrate/glass slide (FIG. 3a-iii). Pressure is added to ensure complete contact between the PDMS and adhesive polymer.
9. The device is then placed into a 120 degrees' Celsius oven for 90 minutes (FIG. 3a-iv).

### Multiple-Layered Devices

The methods disclosed herein enable production of multiple-layered devices that can either be permanently or reversibly assembled. Using reversible bonding methods, various layers of a multiple-layered device can be cleanly disassembled and reused, wherein there is no loss in bond strength in subsequent devices that contain recycled component layers. Using irreversible bonding methods, multiple layered devices can be assembled that are capable of withstanding high pressures.

### Post-Functionalization Adhesion Step Omitted

Selective micropatterning can be performed on the adhesive polymer substrate prior to sealing in the micropatterned surface within a microfluidic device. In this way, the micropatterned areas are not affected by an additional adhesion layer that would otherwise be required to attach layers of a device together. By using the reversible and/or irreversible seal methods disclosed herein to provide self-adhesive, high pressure seals, it is possible to create patterned functionalized surfaces within a microfluidic chamber without any need for adhesives or a clean room, or any extra steps thereby required.

Micropatterning techniques, such as microcontact printing, can be used to create functionalized patterns prior to sealing. Moreover, because the adhesive polymer is used as the substrate to bond PDMS devices, existing designs can easily be integrated with micropatterned surfaces. Due to the characteristic adhesiveness of the substrate, the PDMS chip can seal over any excess patterned area, allowing for a larger tolerance for device alignment. Thus, it is possible to create different patterns over a larger area without concern for alignment or bonding, making it simpler to integrate micropatterned cell culturing with microfluidics.

By comparison, with conventional plasma bonding methods, surface activation of the PDMS is time sensitive, and therefore alignment and bonding of each layer must be done immediately upon activation, typically in a single attempt.

### Example Process

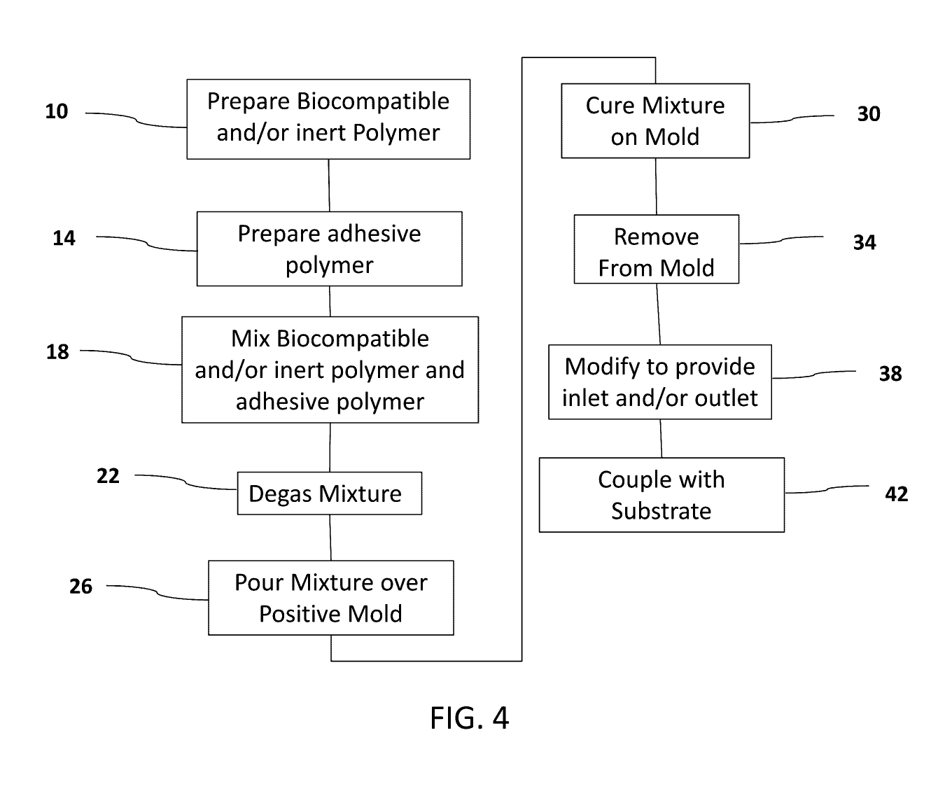

FIG. 4 shows an example of a fabrication of the microfluidic chips using the self-adhesive polymer is as follows. In a step 10, PDMS (Sylgard 184) is prepared by mixing the cross-linker and the base at a ratio of 1:10 by weight. PDMS is one example of an inert and/or a biocompatible silicone. Other silicone based polymer and other inert and/or a biocompatible can be used. In a step 14, a soft skin adhesive polymer (MG7-9850) is made by mixing the part A and part B components ratio of 1:1 by weight. Other adhesive polymers could be used. Thereafter the PDMS (or other biocompatible and/or inert polymer) and MG7-9850 (or other adhesive polymer) are mixed together at a selected ratio. One such ratio is 1:40, for example. Mixing creates an uncured self-adhesive polymer. In a step 22 the uncured self-adhesive polymer is allowed to degas, for example by being placed in vacuum for 10 minutes.

In a step 26, the uncured self-adhesive polymer is poured over a mold, e.g., a positive channel mold. This step can optionally include setting the poured uncured polymer and mold in a vacuum for an additional 15 minutes.

In a step 30, the molded, uncured self-adhesive polymer is cured. For example, the molded uncured polymer can be place into an oven heated to 60° C. to be cured for two hours. In a step 34, the cured self-adhesive polymer is removed from the oven and from the mold. The cured polymer can be cut out, peeled from the mold, and/or cut to size.

The formed device can then be modified in a step 38 to allow a sample to be introduced and to flow therein. For example, an inlet opening and, optionally, an outlet opening can be formed, e.g., punched with a hole puncher. Next, a glass slide is clean and the cured self-adhesive polymer is placed (channel side facing down) onto the glass slide and allowed to seal. Next in a step 42, the formed device can be coupled with a substrate e.g., by application of light pressure to one or both of the formed device and the substrate, to fully secure and/or enclose at least part of the channels.

The devices are used as normal microfluidic devices after their fabricated. We used a previously described procedure with shape memory polymers to create the mold; however, the fabrication steps indicated above can also be used with traditional molding techniques.

### Example Apparatuses

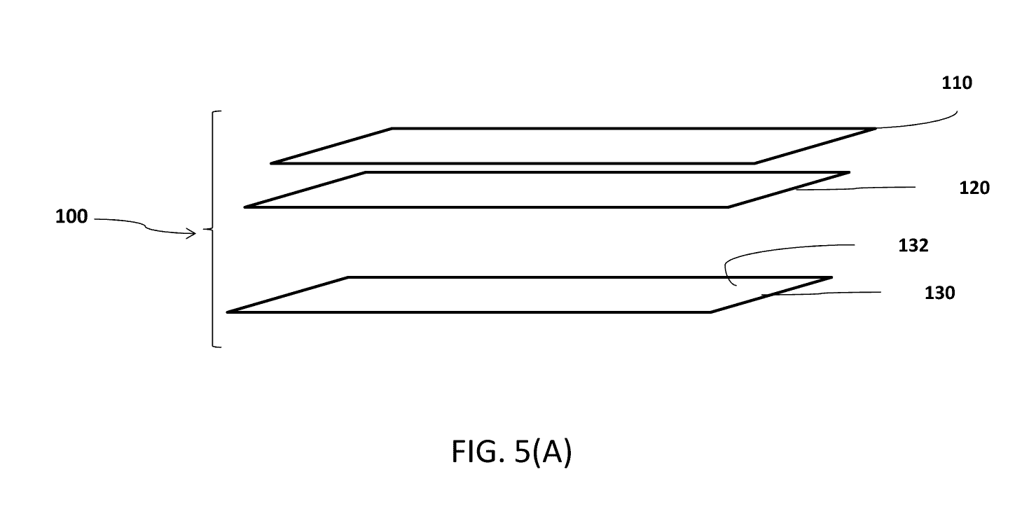

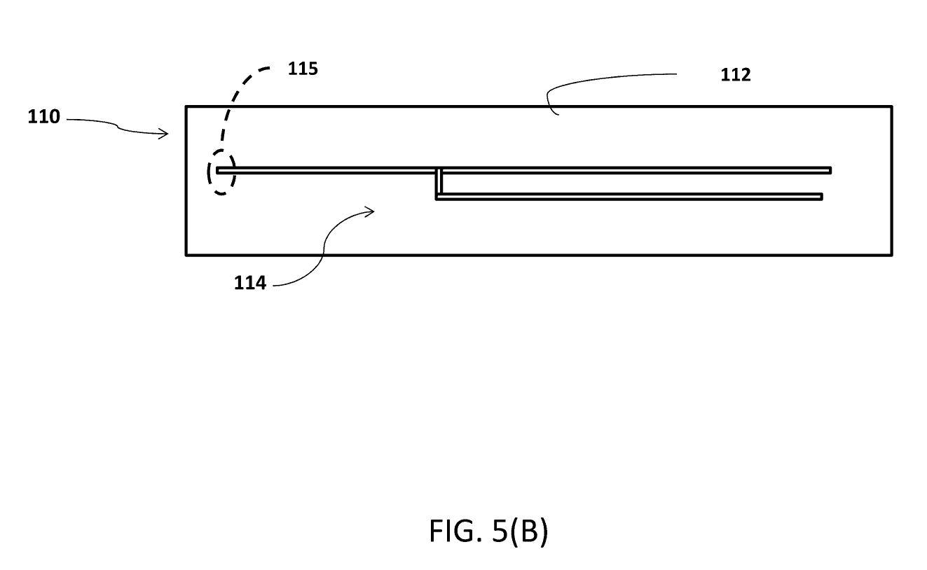

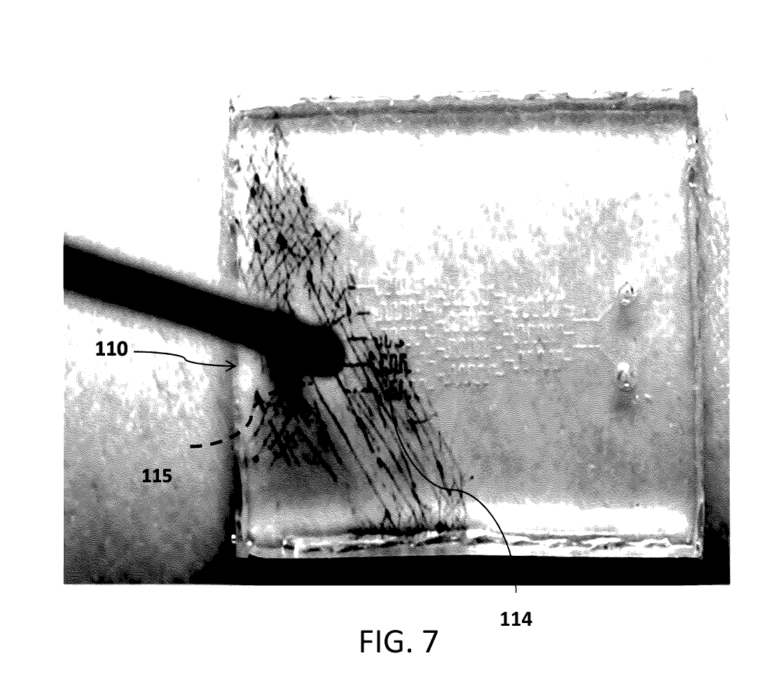

FIG. 5, (a) and (b) show a microfluidic sample handling apparatus 100 and components thereof. The microfluidic sample handling apparatus 100 includes a microfluidic device layer 110 formed according to the methods herein. The microfluidic device layer 110 has, e.g., is made from, an inert polymeric material and a self-adhesive polymer, such as PDMS as discussed above. The microfluidic sample handling apparatus 100 also includes a substrate 130 in some embodiments. In other embodiments, the sample handling apparatus 100 can be configured to be backed by another structure such as directly by the skin of a user or patient as illustrated in FIG. 7. In other embodiments, the microfluidic device layer 110 or a sensing layer with or without a channel or chamber is provided without a substrate. In these embodiments, the user can provide the substrate, which can be a glass slide or even skin if a direct biomedical application is used. Biomedical applications can include collecting sweat using channels or chambers. Biomedical applications can include brining sensors into contact with or adjacency with the skin. The microfluidic device layer 100 can be formed using the methods described herein, e.g. in connection with FIG. 4. The substrate 130 can be formed of glass or another structure that is convenient for flowing a sample through the apparatus 100.

FIG. 5(a) shows that the microfluidic device has a first exposed face 112. The exposed face 112 has at least one channel 114 disposed therein. In other embodiments, the layer 110 comprises a sensing apparatus in addition to or in lieu of the microfluidics. Example sensing apparatuses include an EKG sensor for sending heart parameter, EMG sensor for sensing muscular activity and parameter, and an EEG sensor for sensing brain activity and parameters. The channel 114, if present, can comprise at least one concave area recessed from the face 112 into the thickness of, e.g. the body of the layer 110. The lowest part of the concavity is disposed between the exposed face 112 and a side of the layer 110 opposite the exposed face. The channel is recessed into the body of the layer 110 in a direction away from the first exposed face 112. In some embodiments, the channel is not a recessed area, but is a fluid motive force that can be provided by a surface property, such as a wettability gradient, e.g., superhydrophobicity.

The microfluidic device layer 100 can include a passage 115 extending from a side of the microfluidic device layer opposite the exposed face 112 through the microfluidic device layer 110 and into the channel 114. The passage 115 can form an inlet or an outlet to the microfluidic sample handling apparatus 100. The microfluidic device layer 110 can have more than one such passage, e.g., can have two passages comprising an inlet and an outlet.

The substrate 130 has a second exposed face 132. The first exposed face 112 is configured to adhere directly to the second exposed face 132 in order to enclose the at least one channel 114.

A protective layer 120 can be provided that cover an exposed face 112 of the microfluidic device layer 110. The protective layer 120 can act as a cover. The protective layer 120 can comprise a film. The film can be configured to release prior to use, e.g., be a peelable film.

### Example Uses

FIG. 6(a) shows the layer 110 adhered to the substrate 130. Direct adhesion to the substrate 130 is advantageous in that it eliminates extra adhesives that may be toxic or costly. In the direct skin applications discussed herein, the absence of such components can make the interaction less traumatic to the patient. FIG. 6(b) show the fluid flowing in the left side portion of the channel 114. The self-adhesive polymer has the added advantage of being reusable. The self-adhesive polymer can be peeled off, as illustrated in FIG. 6(c), washed with 70% Ethanol, and air dried. Afterwards the self-adhesive polymer layer can be reattached to a clean glass slide and reused, as illustrated in FIG. 6(d). FIG. 6(e) illustrates that the self-adhesive layer can still adhere even after use. Because the self-adhesive polymer can be removed, it can potentially allow for a more efficient extraction of samples from the channels.

By mixing in a soft skin adhesive, MG7-9850, with standard PDMS at an selected ratio, we are able to successfully create microfluidic devices and other devices including skin adhesive layer apparatuses. In comparison with other microfluidic application processes, our process is less expensive, more efficient, and faster. We do not require specialized equipment or clean room access. It is also less time-consuming because we can apply the self-adhesive polymer in one step without an additional post bake and without requiring additional components such as other adhesives. Other process include additional cure or bake time to effectively seal the device. Moreover, our devices are multi use as we are able to peel the device from substrate, clean it, and re-bond it. The MG7-9850/PDMS mixture is a biocompatible material that allows us to attach microfluidic devices to the skin, further opening up the field to future microfluidic applications. Further, being able to peel it off me also allow for efficient sample extraction from the channels.

Furthermore, the self-adhesive polymer can also interface with standard PDMS microfluidic device fabrication process flow to form an irreversible bond.

FIG. 7 shows an example use of a skin adhesive layer comprising the microfluidic device layer 110 having channels 114 disposed therein. The blue (dark) pattern extending away from the passage 115 indicates fluid flowing in the channels. In variations, the channels 114 are coupled at their end with a collection space, such as a chamber. In other variations, the channels 114 are supplemented by or replaced with a sensor for detecting physiological parameters.

### Example 1

### Plasma-Free Reversible and Irreversible Microfluidic Bonding

We demonstrate a facile, plasma free, process to fabricate both reversibly and irreversibly sealed microfluidic chips using a PDMS-based adhesive polymer mixture. This is a versitile method that is compatible with current PDMS microfluidics processes. It allows for easier fabrication of multilayer microfluidic devices and is compatible with micropatterning of proteins for cell culturing. When combined with our Shrinky-Dink microfluidic prototyping, complete microfluidic device fabrication can be performed without the need for any capital equipment, making microfluidics accessible to the classroom.

### Device Fabrication and Bonding

The adhesive polymer is a mixture of a silicone-based soft skin adhesive (MG 7-9850, Dow Corning®) and traditional PDMS (Sylgard 184, Dow Corning®). Both polymers were first mixed separately and then combined to form the final adhesive mixture. The PDMS was prepared by mixing the cross linker and base at a 1:10 ratio by weight, and the soft skin adhesive was prepared by mixing part A and part B components at a 1:1 ratio by weight. The adhesive polymer mixture was then formed by combining the uncured PDMS and soft skin adhesive at a 1:40 ratio, respectively, by weight. Next, the final adhesive mixture was used to mold the microfluidic devices following the traditional replica molding process (D. C. Duffy, J. C. McDonald, O. J. A. Schueller and G. M. Whitesides, Anal. Chem., 1998, 70, 4974), cured, and bonded to a glass substrate for a reversible seal (FIG. 2a). Alternatively, for an irreversible bond, cured adhesive polymer was spun coat onto a glass slide and cured; the cured adhesive polymer was then used as a substrate to bond traditionally molded PDMS devices (FIG. 2b). The PDMS device was placed directly on the cured adhesive polymer substrate, and heat treated to create an irreversible seal.

### Burst Pressure Test

The bond strength of the interface was measured via a burst pressure test (C. S. Thompson and A. R. Abate, Lab Chip, 2013, 13, 632) for three different conditions: PDMS device to glass substrate (control), adhesive polymer device to glass substrate, and PDMS device to adhesive polymer substrate. For each condition, the pressure within a 3 mm diameter chamber was increased incrementally until failure occurred. The master mold for the chambers were fabricated by adhering 3 mm diameter circles, cut from Frisket Film (Grafix®), onto a flat PMMA surface. Afterwards, either PDMS or the adhesive polymer was poured into the molds, degassed for 15 minutes, and cured for at least 3 hours. The glass substrates were prepared by drilling inlet holes through cleaned glass slides; afterwards, commercially made press fit tubing connectors (Grace Bio-Labs, Inc.) were then adhered over the holes to serve as inlets for the tubing. The adhesive polymer substrate was fabricated by spin coating an additional layer of the 1:40 ratio adhesive polymer on the glass substrate at 800 rpm for 60 seconds and allowed to fully cure. Afterwards, the inlet holes were cleaned.

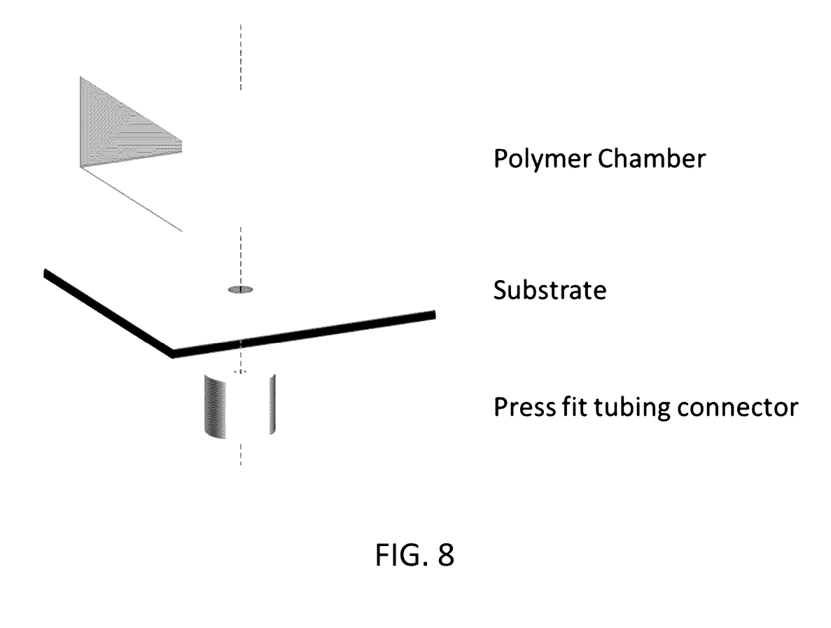

Device assembly occurred by placing the cured device chamber side down onto the substrate so that the center aligned with the inlet and press fit tubing (FIG. 8). Slight pressure was applied to ensure full contact between both surfaces. The devices were then heat treated in an oven at 120 degrees Celsius for 90 minutes.

The burst pressure test set up consisted of a closed tubing system that connected the 3 mm chamber to a 20 ml syringe and digital manometer (Dwyer Series 490). The pressure of the system was controlled using a syringe pump, which decreased the volume of the syringe by 0.5 ml intervals at a rate of 2 ml/min. Measurements were taken once the pressure equilibrated; the last stable pressure before bond failure for each device was reported. To determine reusability, three separate burst pressure measurements were taken for the same set of adhesive polymer devices bonded to the glass. After each test, the adhesive polymer was removed from the glass slide, washed with isopropyl alcohol, and dried in an oven at 60 degrees Celsius for 30 minutes. The glass substrate was also cleaned in the same manner. Both the glass slide and adhesive polymer device were additionally cleaned with Scotch® tape 3 times between testing.

### Swell Test

To compare the degree of swelling between traditional PDMS and the adhesive polymer, a swell study was done with five different solvents. Solid squares of the adhesive polymer and traditional PDMS, respectively, were made using the same replica molding process as described above. A set of 5 squares was used for each solvent. The pieces were submerged in separate containers and imaged with a DSLR camera (Canon EOS Rebel T3i) while immersed in solvent. After full immersion for 24 hours at room temperature, the pieces were then imaged again. The length of each edge was measured before and after from the digital image using ImageJ software. The solvents examined were acetone, isopropyl alcohol (IPA), ethanol, water, and dimethyl sulfoxide (DMSO).

### Microfluidic Chip Fabrication

To demonstrate the reversible sealing capability of the adhesive polymer, gradient generating devices were fabricated using the Shrinky-Dink procedure, first developed by Grimes et al. (A. Grimes, D. N. Breslauer, M. Long, J. Pegan, L. P. Lee and M. Khine, Lab Chip, 2007, 8, 170), and reused multiple times. AutoCAD® drawings of both designs were printed onto pre-stressed polystyrene (PS) using a laser printer. The PS was then shrunk in an oven at 160 degrees Celsius, allowing the ink to reflow to create rounded protrusions. The adhesive polymer was then poured into the mold, degassed for 15 minutes in vacuum, and cured at 60 degrees Celsius for 2 hours. A thin layer of PDMS was subsequently cured on top to serve as mechanical support for the inlet and outlet tubing insertion. Inlets and outlets were punched through the adhesive polymer and PDMS bilayer using a biopsy punch (Miltex®), and the surface of both the glass slide and the adhesive polymer were cleaned prior to bonding. The devices were placed chamber side down onto the cleaned glass slides and baked at 120 degrees Celsius for 90 minutes. For the gradient generator, the channels were primed with 70% ethanol before flowing blue and yellow food dye at a flow rate of 0.001 μl/min. This process demonstrates that the entire microfluidic device can be made without any capital equipment or clean room access.

A multilayer micromixer was fabricated by stacking alternating layers of PDMS and adhesive polymer (FIG. 1). The positive mold for each layer was fabricated by laser cutting the outline of the channel shape in Frisket Film adhered onto a flat PMMA sheet. Afterwards, the Frisket Film surrounding the channel was removed leaving only the positive channel structure. PDMS was used to mold the first and third layer of the device while the adhesive polymer was used to mold the middle layer. Once fully cured, the negative mold was then released, and inlet and outlet holes were punched using a biopsy punch. The device was then assembled onto a glass slide laminated with a layer of pre-cured adhesive polymer; each layer of PDMS and adhesive polymer were stacked sequentially, with the first layer adhered onto the pre-cured adhesive polymer glass slide. Slight pressure was applied and the construct was heated for 90 minutes at 120 degrees Celsius. Afterwards, the channels were primed with 70% ethanol, and food dye were flowed through the inlets. As can be seen from FIG. 1a, blue and yellow food dye were individually flowed through the first and second layer of the multilayer chip; the two food dye mixed in the vertical column connecting all three layers before flowing through the third layer. FIG. 1b shows the exploded view of the multilayer chip with the inlet and outlet holes aligned.

### Cell Patterning and Culture

To show the facile integration of micropatterning within a fluidic device, cell patterning was performed by plating human stem cell-derived cardiomyocytes (hES2-7E) on the adhesive polymer within a PDMS fluidic chamber. A large oblong shaped fluidic chamber with a height of 1.52 mm was molded using PDMS; inlet and outlet holes were punched into opposite corners. A thin layer of the adhesive polymer was then spun coat onto a microscope slide, and allowed to cure at 60 degrees Celsius for 3 hours. Traditionally, silicone polymers display poor cell adhesion due to the materials' high surface hydrophobicity (Y. J. Chuah, Y. T. Koh, K. Lim, N. V. Menon, Y. Wu and Y. Kang, Scientific Reports, 2015, 5, 18162). Pruitt et al. demonstrated that proteins necessary for cell adhesion can be covalently bonded to PDMS via an organosilane process using 3-glycidoxypropyltrimethoxysilane (GPTMS) (A. J. S. Ribeiro, K. Zaleta-Rivera, E. A. Ashley and B. L. Pruitt, ACS Appl. Mater. Interfaces, 2014, 6, 15516). The adhesive polymer on the microscope slides were plasma treated with oxygen for 3 minutes and then incubated in a methanol solution of 20% GPTMS. To pattern in a deterministic manner, a shadow mask was applied to the adhesive polymer prior to the plasma treatment. Following the organosilane treatment, the surface was sealed by placing the PDMS chamber on top. The construct was then sterilized via autoclave, in which the high temperature helps to strengthen the bond between the PDMS and the adhesive polymer. After sterilization, Matrigel (Corning®) was flowed into the construct. Cardiomyocytes were then loaded at a density of [symbol] 6.3×105 cells/ml. The contractility was confirmed and quantified with an optical flow-based method (E. K. Lee, Y. K Kurokawa, R. Tu, S. C. George and M. Khine, Scientific Reports, 2015, 5, 11817).

### Results and Discussion

### Characterization of Bond Strength and Swelling

The soft skin adhesive is a FDA-approved, PDMS-based platinum catalyzed elastomer. By introducing varying amounts of standard PDMS to the soft skin adhesive, the stiffness and tackiness of the polymer can be tuned. The adhesive nature of the polymer was leveraged as the bonding mechanism for sealing the device to the substrate through direct contact. A 1:40 ratio of the PDMS to the soft skin adhesive was found to have an optimal stiffness for molding while maintaining enough adhesion to bond to glass. However, the ratio can also be adjusted for other applications.

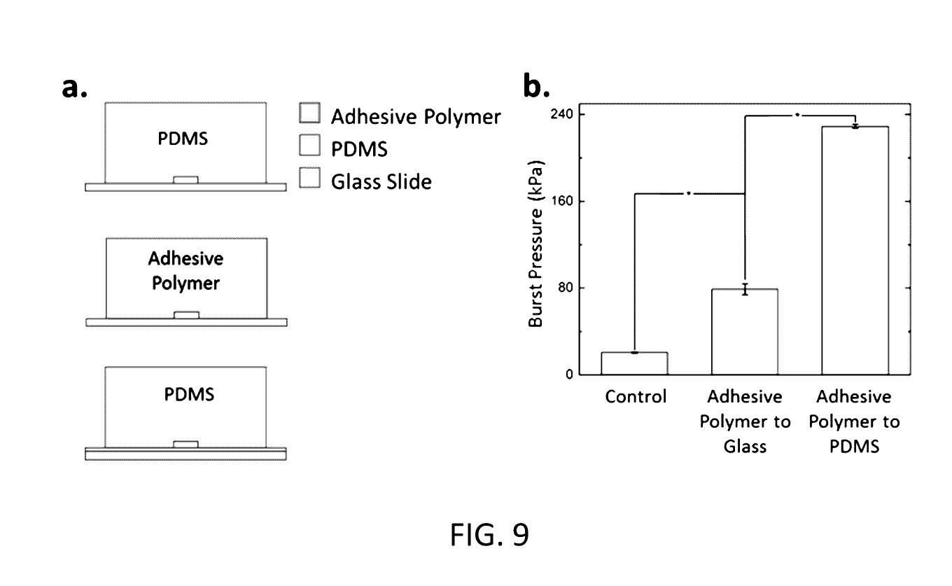

The adhesive polymer formed a reversible, bond when placed directly onto an untreated glass substrate; this bond is stronger than that of the PDMS control. FIG. 9a shows a cross-sectional schematic of the control, reversible, and irreversible conditions. Despite the increased bond strength, the polymer can still be reversibly removed without harming the channel footprint. The bond between the adhesive polymer and glass failed after 79±5 kPa. As seen in FIG. 9b, this failure pressure is fourfold higher than that of the PDMS control, which failed after 21±1 kPa. Failure of the bonds occurred via concentric delamination from the edge of the chamber outward towards the edge of the chip. Post removal, the adhesive polymer chambers were then washed and re-bonded to a glass substrate for reuse. We found no significant loss in the burst pressure with subsequent reuse of the devices (Table 1). This bond strength is sufficient for many microfluidic applications including: gradient generation, droplet generation, and cell culturing (B. J. Adzima and S. S. Velankar, J. Micromech. Microeng., 2006, 16, 1504; R. Gomez-Sjoberg, A. A. Leyrat, D. M. Pirone, C. S. Chen and S. R. Quake, Anal. Chem., 2007, 79, 8857; and V. VanDelinder and A. Groisman, Anal. Chem., 2006, 78, 3765).

TABLE 1
Repeated burst pressure measurements for same set
of adhesive polymer.

| Trial | 1 | 2 | 3 |
|---|---|---|---|
| Burst Pressure | 79 ± 5 kPa | 76 ± 4 kPa | 77 ± 3 kPa |

There was no significance between each trial (<0.001).

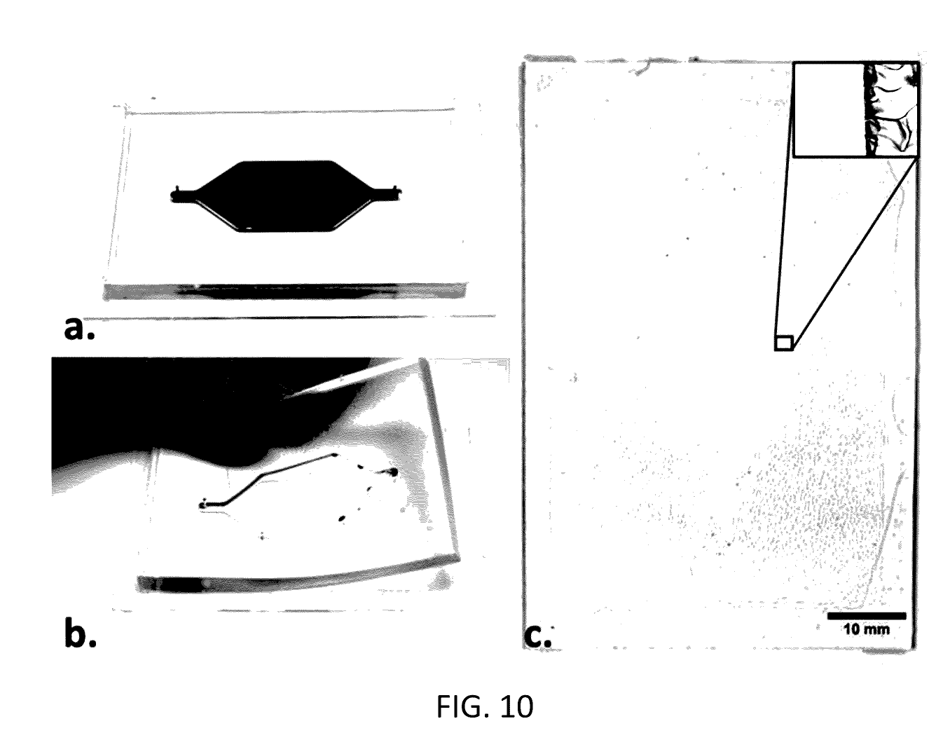

Alternatively, an irreversible seal can also be achieved by bonding cured PDMS to a cured adhesive polymer substrate. As previously stated, Sia et al. defines irreversibly sealed devices capable of withstanding 207-345 kPa (S. K Sia and G. M. Whitesides, Electrophoresis, 2003, 24, 3563); the bond strength between the PDMS device and adhesive polymer substrate was able to withstand a pressure of 229±2 kPa (FIG. 9b). In fact, bond failure did not occur at this point, but, rather, the upper limit of the manometer was reached. Moreover, there was no visual indication of delamination at this pressure, and subsequent removal of the PDMS chambers tore the adhesive polymer substrate. The boundary between the bonded region and the chamber of the substrate post-device removal is indicated in the inset image of FIG. 10, which shows a top down view taken using a 3D laser scanning microscope (Keyence VK-X 100 series); the bonded region was torn during the removal, while the chamber region remained undisturbed. Consequently, the adhesive polymer is softer than PDMS, and when a tensile stress is applied to remove the PDMS device, the adhesive substrate mechanically fails before the PDMS does. Although the PDMS device cannot be reused afterwards, this method provides a simple way for device removal by leveraging the adhesive polymer as a sacrificial layer. Moreover, this method is compatible with current microfluidic fabrication using PDMS replica molding and eliminates the need for oxygen plasma treatment.

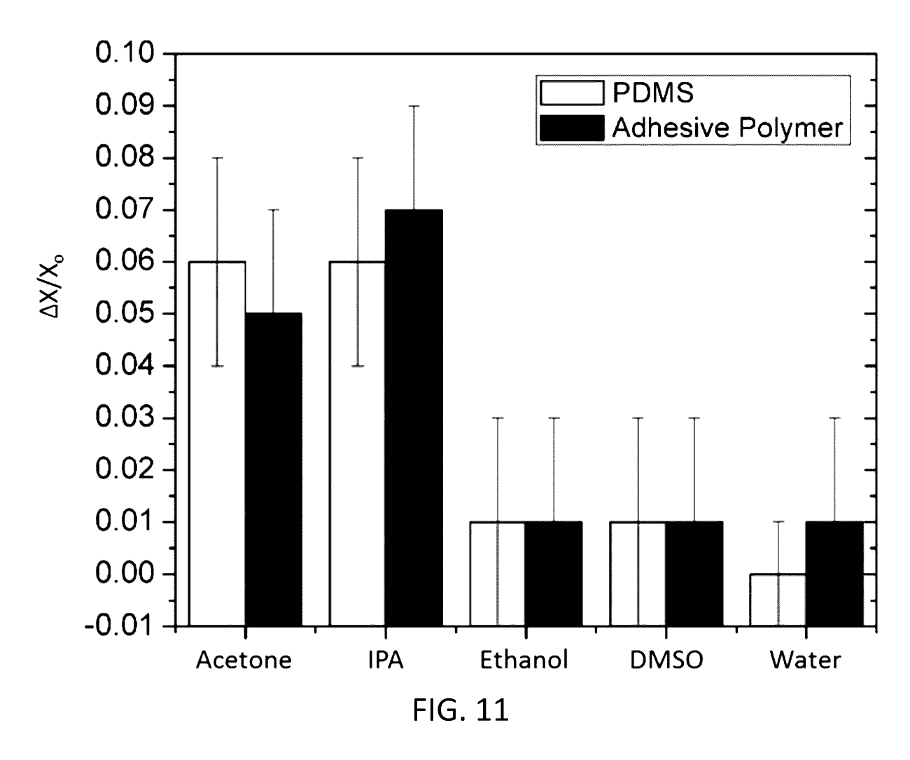

There was no significant difference in swelling between the PDMS and adhesive polymer for all the solvents tested (FIG. 11), suggesting that the swelling behavior of the adhesive polymer is similar to PDMS. The solvents chosen were the most commonly found in a standard laboratory, and moreover, often used in cell culture protocols. The pieces were found to have swelled the most in IPA, followed closely by acetone; the swelling in the other solvents tested was found to be negligible. However, even the most significant swelling remained at 7% or below, making the adhesive polymer suitable for standard use within a common lab.

### Gradient Generator

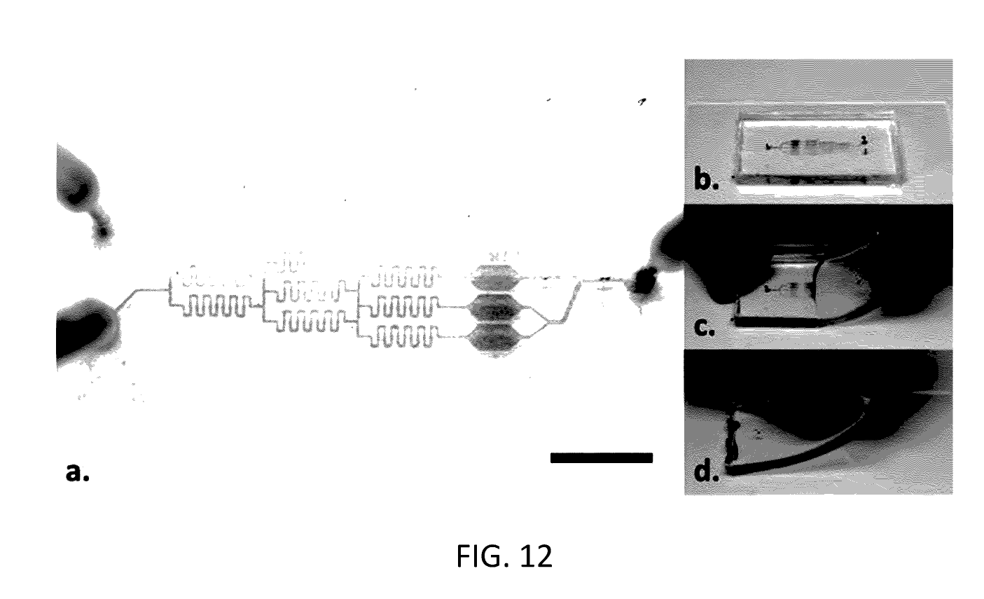

A concentration gradient was created by reversibly bonding an adhesive polymer gradient generator device to glass (FIG. 12a). The channel height and width were approximately 32 μm and 180 μm, respectively, and blue and yellow food dye was flowed through the inlet to generate the gradient. After the initial operation, the gradient generator was removed, cleaned, and re-bonded. FIG. 12 (b-d) show the step-wise removal of the gradient generator from the glass slide after the second use. While the adhesive polymer can still mold conventional micron-sized channels, the polymer itself is still softer than PDMS. Thus, channels with lower aspect ratios will be more likely to deform and collapse onto the substrate with applied pressure. However, the adhesive polymer stiffness can be optimized to mold lower aspect ratio geometries.

### 3D Microfluidics

Mixing in a 2D microfluidic environment is difficult to achieve due to the natural laminar flow regime of the small channel; however, this problem can be alleviated by introducing a 3D geometry that disrupts the laminar flow (R. H. Liu, M. A. Stremler, K. V. Sharp, M. G. Olsen, J. G. Santiago, R. J. Adrian, H. Aref and D. J. Beebe, Journal of Microelectromechanical Systems, 2000, 9, 190; and C.-S. Chen, D. N. Breslauer, J. I. Luna, A. Grimes, W.-C. Chin, L. P. Lee and M. Khine, Lab Chip, 2008, 8, 622). The 3D microfluidic chip is a three-layer micromixer interconnected with holes punched through each layer. The device consists of two inputs that allow fluid flow to travel through two separate layers before mixing and exiting through the last layer; in other words, blue and yellow food dye flowed through the first and second layer, individually, before mixing and flowing through the third layer. The layers are bonded irreversibly together by having alternate layers of PDMS and adhesive polymer.

Moreover, because the PDMS and adhesive polymer will not irreversibly bond until heat treated, this fabrication process allows for multiple attempts to position each layer. If the initial placement is not fully aligned, then the device can be removed and realigned. With the traditional plasma bonding method, the surface activation of the PDMS is time sensitive, and therefore the alignment and bonding of each layer must be done immediately upon activation, typically in a single attempt. As the adhesive polymer and PDMS do not irreversibly bond until after prolonged exposure to heat, multiple alignment attempts can be made for each layer without a significant effect on the bond.

### Cell Patterning

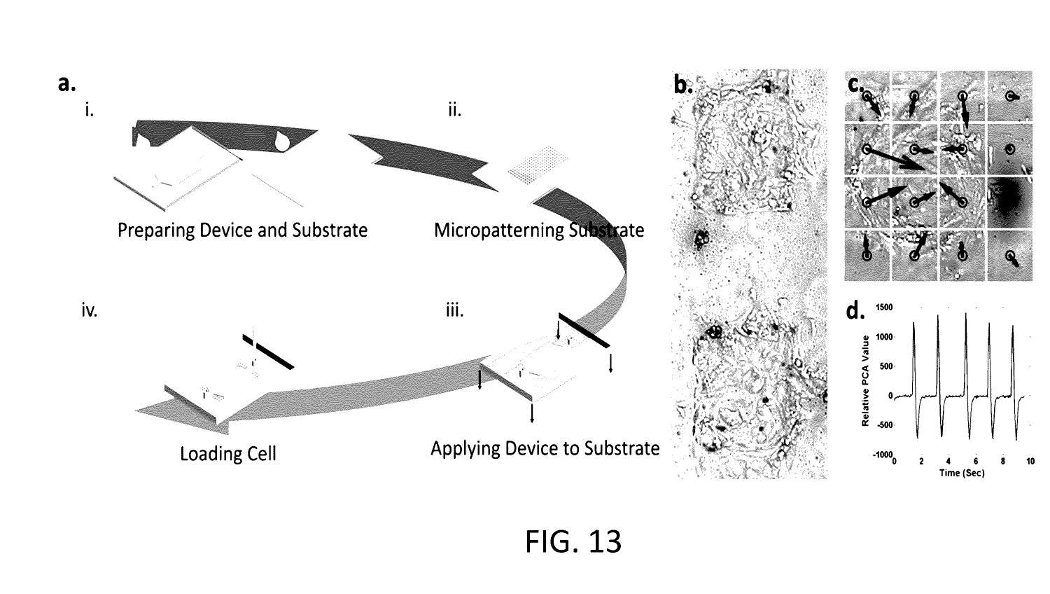

A large patterned square array was created on the adhesive polymer prior to sealing the microfluidic chip. As seen in FIG. 13a, functionalization of the surface for adhesion occurs right before the fluidic component is sealed over the substrate. Afterwards, human stem cell-derived cardiomyocytes were loaded and patterned on the substrate within the fluidic chamber. FIG. 13b shows two square islands of cardiomyocytes patterned on the substrate. Contractility was assessed using an optical flow based method, which generates motion vectors following the cardiomyocyte's contraction and relaxation, as seen in FIG. 13c principal component analysis (PCA) was then used to summarize the motion vectors generated from the optical flow into one variable that automatically discerns the contraction and relaxation phase of a contractile event (FIG. 13d). Contractility was evident within two days of cell seeding, and the cells were viable up to 150 days. Additionally, as discussed above, the PDMS chamber can still be easily removed from the adhesive polymer layer for easy access to the cells. FIG. 10 shows the fluidic chamber and the subsequent removal of the device from the substrate.

We demonstrated that selective micropatterning can be performed on the adhesive polymer substrate and then sealed by a microfluidic device in a facile manner. The micropatterned areas are not affected by an additional adhesion layer (see process flow in FIG. 13a). Accordingly, other micropatterning techniques such as microcontact printing can be used to create functionalized patterns prior to sealing. Moreover, because the adhesive polymer is used as the substrate to bond PDMS devices, existing designs can easily be integrated with micropatterned surfaces. Due to the characteristic adhesiveness of the substrate, the PDMS chip can seal over any excess patterned area, allowing for a larger tolerance for device alignment. Thus, it is possible to create different patterns over a larger area without concern for alignment or bonding, making it simpler to integrate micropatterned cell culturing with microfluidics.

### CONCLUSIONS

We demonstrated a simple and versatile system for fabricating both reversibly and irreversibly sealed microfluidic chips. While the adhesive polymer used in this Technical Innovation demonstrates similar properties to PDMS, and we have successfully cultured fragile hESC-CM with this material for >150 days, further characterization is ongoing. However, the polymer shows promise in simplifying the fabrication procedure for PDMS-based devices.

Use of the adhesive polymer can be easily integrated into the standard PDMS soft lithographic process flow, simplifying the fabrication procedure while also allowing for higher throughput. When combined with the Shrinky-Dink microfluidic rapid prototyping method, fabrication of a completed microfluidic device can be accomplished from start to finish without the need for specialized equipment, such as an oxygen plasma machine, or a cleanroom. This allows for microfluidics in a classroom or low resource setting area. This bonding method also enables simple fabrication of 3D microfluidic devices. Moreover, certain micropatterning techniques can be directly integrated into the fabrication procedure. Importantly, this process allows researchers and teachers who are not in specialized microfluidic laboratories, such as those in the biological field, to be able to fabricate and implement a microfluidic platform in a low cost and simple manner.

While the present description sets forth specific details of various embodiments, it will be appreciated that the description is illustrative only and should not be construed in any way as limiting. Furthermore, various applications of such embodiments and modifications thereto, which may occur to those who are skilled in the art, are also encompassed by the general concepts described herein. Each and every feature described herein, and each and every combination of two or more of such features, is included within the scope of the present invention provided that the features included in such a combination are not mutually inconsistent.

Some embodiments have been described in connection with the accompanying drawings. However, it should be understood that the figures are not drawn to scale. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, it will be recognized that any methods described herein may be practiced using any device suitable for performing the recited steps.

For purposes of this disclosure, certain aspects, advantages, and novel features are described herein. It is to be understood that not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Although these inventions have been disclosed in the context of certain preferred embodiments and examples, it will be understood by those skilled in the art that the present inventions extend beyond the specifically disclosed embodiments to other alternative embodiments and/or uses of the inventions and obvious modifications and equivalents thereof. In addition, while several variations of the inventions have been shown and described in detail, other modifications, which are within the scope of these inventions, will be readily apparent to those of skill in the art based upon this disclosure. It is also contemplated that various combination or sub-combinations of the specific features and aspects of the embodiments may be made and still fall within the scope of the inventions. It should be understood that various features and aspects of the disclosed embodiments can be combined with or substituted for one another in order to form varying modes of the disclosed inventions. Further, the actions of the disclosed processes and methods may be modified in any manner, including by reordering actions and/or inserting additional actions and/or deleting actions. Thus, it is intended that the scope of at least some of the present inventions herein disclosed should not be limited by the particular disclosed embodiments described above. The limitations in the claims are to be interpreted broadly based on the language employed in the claims and not limited to the examples described in the present specification or during the prosecution of the application, which examples are to be construed as non-exclusive.

What is claimed is:

1. A method of forming a microfluidic device, comprising:
combining a volume of uncured liquid silicone based polymer with a volume of adhesive polymer to provide a flowable material;
applying the flowable material to a mold and curing the flowable material on the mold to form a microfluidic device layer comprising an exposed face with at least one channel or chamber; and
contacting the exposed face of the microfluidic device layer to a substrate to adhere the microfluidic device layer to the substrate to enclose the at least one channel or chamber to form a microfluidic device.

2. The method of claim 1, wherein a ratio of the volume of the uncured liquid silicone based polymer to the volume of the adhesive polymer is at least 1:10, 1:20, 1:30, 1:40, 1:60 and/or does not exceed 1:100.

3. The method of claim 1, wherein the mold comprises a positive mold.

4. The method of claim 1, wherein the curing comprises heating the flowable material.

5. The method of claim 4, wherein the heating comprises heating for 2 hours at 60° C.

6. The method of claim 1, wherein the curing comprises applying a vacuum to the flowable material.

7. The method of claim 1, wherein the silicone comprises PDMS and the adhesive polymer comprises a soft-skin adhesive.

8. The method of claim 1, further comprising forming an inlet to the microfluidic device by creating a passage through at least one of the microfluidic device layer and the substrate.

9. The method of claim 8, wherein the passage is created through the microfluidic device layer prior to adhering the exposed face to the substrate.

10. The method of claim 1, wherein the substrate is micropatterned to create functionalized patterns on the substrate to contacting the exposed face of the microfluidic device layer to a substrate.

11. The method according to claim 10, wherein the substrate is micropatterned by microcontact printing.

12. A method of forming a microfluidic device, comprising:
combining a volume of uncured liquid silicone based polymer with a volume of adhesive polymer to provide an intermediary material;
applying a layer of the intermediary material to a substrate and curing the layer of the intermediary material on the substrate;
obtaining a silicon based polymer that comprises an exposed face that comprises at least one channel or chamber; and
contacting the exposed face of the silicon based polymer to the cured layer of the intermediary material, wherein the exposed face of the silicon based polymer adheres to the cured layer of the intermediary material to enclose the at least one channel or chamber to form a microfluidic device.

13. The method of claim 12, wherein a ratio of the volume of the uncured liquid silicone based polymer to the volume of the adhesive polymer is at least 1:10, 1:20, 1:30, 1:40 or 1:60 and/or does not exceed 1:100.

14. The method of claim 12, wherein the mold comprises a positive mold.

15. The method of claim 12, wherein the curing comprises heating the layer of the intermediary material on the substrate.

16. The method of claim 15, wherein the heating comprises heating for 2 hours at 60° C.

17. The method of claim 12, wherein the curing comprises applying a vacuum to the layer of the intermediary material on the substrate.

18. The method of claim 12, wherein the silicone comprises PDMS and the adhesive polymer comprises a soft-skin adhesive.

19. The method of claim 12, further comprising forming an inlet to the microfluidic device by creating a passage through at least one of the silicon based polymer nd the substrate.

20. The method of claim 19, wherein the passage is created through the silicon based polymer prior to adhering the exposed face to the substrate.

21. The method of claim 12, wherein the substrate layer is micropatterned to create functionalized patterns on the substrate prior to contacting the exposed face of the silicon based polymer to a substrate.

22. The method according to claim 12, wherein the substrate is micropatterned by microcontact printing.

23. A microfluidic device comprising:
a first substrate layer, and
a second layer comprising a silicone based polymer and an adhesive polymer, wherein the second layer comprises at least one channel or chamber at a surface of the second layer,
wherein the first substrate layer and the second layer are adhered together to enclose the at least one channel or chamber within the microfluidic device.

24. A microfluidic device comprising:
a first substrate layer,
a second intermediary layer that comprises a silicone based polymer and an adhesive polymer, and
a third layer comprising a silicon based polymer that comprises at least one channel or chamber at a surface of the third layer;
wherein the first substrate layer is adhered to the second intermediary layer and the third layer is adhered to the second intermediary layer, wherein the at least one channel or chamber at the surface of the third layer is enclosed within the microfluidic device.

25. A sensor comprising a microfluidic device layer comprising a silicone based polymer and an adhesive polymer, the microfluidic device layer comprising an exposed face that is configured to adhere directly to skin of a user or patient.

26. The sensor according to claim 25, wherein when the microfluidic device is placed on a skin surface, fluid in a channel in the microfluidic device does not contact the skin surface.

27. The sensor according to claim 25, wherein when the microfluidic device is placed on a skin surface, fluid in a channel in the microfluidic device does not contact the skin surface.
